(19) United States
(12) Patent Application Publication
(10) Pub. No.: US 2013/0224228 A1
(43) Pub. Date: Aug. 29, 2013

(54) Antibody-Drug Conjugates and Related Compounds, Compositions, and Methods

(71) Applicant: Igenica, Inc., (US)

(72) Inventors: David Y. Jackson, Belmont, CA (US); Edward Ha, San Francisco, CA (US)

(73) Assignee: Igenica, Inc., Burlingame, CA (US)

(21) Appl. No.: 13/705,074

(22) Filed: Dec. 4, 2012

Related U.S. Application Data
(60) Provisional application No. 61/566,909, filed on Dec. 5, 2011.

(51) Int. Cl. A61K 39/395

(52) U.S. Cl. CPC A61K 39/39558
USPC 424/179.1; 530/391.9; 546/256; 530/331; 530/330

(57) ABSTRACT

Antibody-cytotoxin antibody-drug conjugates and related compounds, such as linker-cytotoxin conjugates and the linkers used to make them, tubulysin analogs, and intermediates in their synthesis; compositions; and methods, including methods of treating cancers.

### CROSS-REFERENCE TO RELATED APPLICATIONS

This invention claims the priority under 35 USC 119(e) of U.S. Provisional Application No. 61/566,909, filed 5 Dec. 2011, “Antibody-drug Conjugates and Methods”, which is incorporated into this application by reference.

### BACKGROUND OF THE INVENTION

1. Field of the Invention

This invention relates to antibody-drug conjugates (ADCs) and related compounds, such as linkers used to make them, tubulysin analogs, and intermediates in their synthesis; compositions; and methods, including methods of treating cancers.

2. Description of the Related Art

Cancer is the second most prevalent cause of death in the U.S, yet there are few effective treatment options beyond surgical resection. Of the medical treatments for cancers, the use of monoclonal antibodies targeting antigens present on the cancer cells has become common. Anticancer antibodies approved for therapeutic use in the USA include alemtuzumab (CAMPATH®), a humanized anti-CD52 antibody used in the treatment of chronic lymphocytic leukemia; bevacizumab (AVASTIN®), a humanized anti-VEGF antibody used in colorectal cancer; cetuximab (ERBITUX®), a chimeric anti-epidermal growth factor antibody used in colorectal cancer, head and neck cancer, and squamous cell carcinoma; ipilimumab (YERVOY®), a human anti-CTLA-4 antibody used in melanoma; ofatumumab (ARZERRA®), a human anti-CD20 antibody used in chronic lymphocytic leukemia; panitumumab (VECTIBIX®), a human anti-epidermal growth factor receptor antibody used in colorectal cancer; rituximab (RITUXAN®), a chimeric anti-CD20 antibody used in non-Hodgkin lymphoma; tositumomab (BEXXAR®), a murine anti-CD20 antibody used in non-Hodgkin lymphoma; and trastuzumab (HERCEPTIN®), a humanized anti-HER2 antibody used in breast cancer. While these antibodies have proven useful in the treatments of the cancers for which they are indicated, they are rarely curative as single agents, and are generally used in combination with standard chemotherapy for the cancer.

As an example, trastuzumab is a recombinant DNA-derived humanized monoclonal antibody that selectively binds with high affinity to the extracellular domain of the human epidermal growth factor receptor2 protein, HER2 (ErbB2) (Coussens et al., Science 1985, 230, 1132-9; Salmon et al., Science 1989, 244, 707-12), thereby inhibiting the growth of HER2-positive cancerous cells. Although HERCEPTIN is useful in treating patients with HER2-overexpressing breast cancers that have received extensive prior anti-cancer therapy, some patients in this population fail to respond or respond only poorly to HERCEPTIN treatment. Therefore, there is a significant clinical need for developing further HER2-directed cancer therapies for those patients with HER2-overexpressing tumors or other diseases associated with HER2 expression that do not respond, or respond poorly, to HERCEPTIN treatment.

Antibody drug conjugates (ADCs), a rapidly growing class of targeted therapeutics, represent a promising new approach toward improving both the selectivity and the cytotoxic activity of cancer drugs. See, for example, Trail et al., “Monoclonal antibody drug immunoconjugates for targeted treatment of cancer”, Cancer Immunol. Immunother. 2003, 52, 328-337; and Chari, “Targeted Cancer Therapy: Conferring Specificity to Cytotoxic Drugs”, Acc. Chem. Res., 2008, 41(1), 98-107. These ADCs have three components: (1) a monoclonal antibody conjugated through a (2) linker to a (3) cytotoxin. The cytotoxins are attached to either lysine or cysteine sidechains on the antibody through linkers that react selectively with primary amines on lysine or with sulfhydryl groups on cysteine. The maximum number of linkers/drugs that can be conjugated depends on the number of reactive amino or sulfhydryl groups that are present on the antibody. A typical antibody contains up to 90 lysines as potential conjugation sites; however, the optimal number of cytotoxins per antibody for most ADCs is typically between 2 and 4 due to aggregation of ADCs with higher numbers of cytotoxins. As a result, conventional lysine linked ADCs currently in clinical development are heterogeneous mixtures that contain from 0 to 10 cytotoxins per antibody conjugated to different amino groups on the antibody. Key factors in the success of an ADC include that the monoclonal antibody is cancer antigen specific, non-immunogenic, low toxicity, and internalized by cancer cells; the cytotoxin is highly potent and is suitable for linker attachment; while the linker may be specific for cysteine (S) or lysine (N) binding, is stable in circulation, may be protease cleavable and/or pH sensitive, and is suitable for attachment to the cytotoxin.

Anticancer ADCs approved for therapeutic use in the USA include brentuximab vedotin (ADCETRIS®), a chimeric anti-CD30 antibody conjugated to monomethylauristatin E used in anaplastic large cell lymphoma and Hodgkin lymphoma; and gemtuzumab ozogamicin (MYLOTARG®), a humanized anti-CD33 antibody conjugated to calicheamicin γ used in acute myelogeneous leukemia—though this was withdrawn in 2010 for lack of efficacy.

Although several ADCs have demonstrated recent clinical success, the utility of most ADCs currently in development may be limited by cumbersome synthetic processes resulting in high cost of goods, insufficient anti-tumor activity associated with limited potency of the cytotoxic drug, and questionable safety due to linker instability and ADC heterogeneity. See, for example, Ducry et al., “Antibody-Drug Conjugates: Linking Cytotoxic Payloads to Monoclonal Antibodies”, Bioconjugate Chem. 2010, 21, 5-13; Chari, “Targeted Cancer Therapy: Conferring Specificity to Cytotoxic Drugs”, Acc. Chem. Res. 2008, 41, 98-107; and Senter, “Recent advancements in the use of antibody drug conjugates for cancer therapy”, Biotechnol.: Pharma. Aspects, 2010, 11, 309-322.

As an example, trastuzumab has been conjugated to the maytansinoid drug mertansine to form the ADC trastuzumab emtansine, also called trastuzumab-DM1 or trastuzumab-MC-DM1, abbreviated T-DM1 (LoRusso et al., “Trastuzumab Emtansine: A Unique Antibody-Drug Conjugate in Development for Human Epidermal Growth Factor Receptor 2-Positive Cancer”, Clin. Cancer Res. 2011, 17, 6437-6447; Burris et al., “Trastuzumab emtansine: a novel antibody-drug conjugate for HER2-positive breast cancer”, Expert Opin. Biol. Ther. 2011, 11, 807-819). It is now in Phase III studies in the US for that indication. The mertansine is conjugated to the trastuzumab through a maleimidocaproyl (MC) linker which bonds at the maleimide to the 4-thiovaleric acid terminus of the mertansine side chain and forms an amide bond between the carboxyl group of the linker and a lysine basic amine of the trastuzumab. Trastuzumab has 88 lysines (and 32 cysteines). As a result, trastuzumab emtansine is highly heterogeneous, containing dozens of different molecules containing from 0 to 8 mertansine units per trastuzumab, with an average mertansine/trastuzumab ratio of 3.4.

Antibody cysteines can also be used for conjugation to cytotoxins through linkers that contain maleimides or other thiol specific functional groups. A typical antibody contains 4, or sometimes 5, interchain disulfide bonds (2 between the heavy chains and 2 between heavy and light chains) that covalently bond the heavy and light chains together and contribute to the stability of the antibodies in vivo. These interchain disulfides can be selectively reduced with dithiothreitol, tris(2-carboxyethyl)phosphine, or other mild reducing agents to afford 8 reactive sulfhydryl groups for conjugation. Cysteine linked ADCs are less heterogeneous than lysine linked ADCs because there are fewer potential conjugation sites; however, they also tend to be less stable due to partial loss of the interchain disulfide bonds during conjugation, since current cysteine linkers bond to only one sulfur atom. The optimal number of cytotoxins per antibody for cysteine linked ADCs is also 2 to 4. For example, ADCETRIS is a heterogeneous mixture that contains 0 to 8 monomethylauristatin E residues per antibody conjugated through cysteines.

The tubulysins, first isolated by the Höfle/Reichenbach group from myxobacterial cultures (Sasse et al., J. Antibiot. 2000, 53, 879-885), are exceptionally potent cell-growth inhibitors that act by inhibiting tubulin polymerization and thereby induce apoptosis. (Khalil et al., Chem. Biochem. 2006, 7, 678-683; and Kaur et al., Biochem. J. 2006, 396, 235-242). The tubulysins, of which tubulysin D is the most potent, have activity that exceeds most other tubulin modifiers including, the epothilones, vinblastine, and paclitaxel (TAXOL®), by 10- to 1000-fold. (Steinmetz et al., Angew. Chem. 2004, 116, 4996-5000; Steinmetz et al., Angew. Chem. Int. Ed. 2004, 43, 4888-4892; and Höfle et al., Pure App. Chem. 2003, 75,167-178). Paclitaxel and vinblastine are current treatments for a variety of cancers, and epothilone derivatives are under active evaluation in clinical trials. Synthetic derivatives of tubulysin D would provide essential information about the mechanism of inhibition and key binding interactions, and could have superior properties as anticancer agents either as isolated entities or as chemical warheads on targeted antibodies or ligands.

Tubulysin D is a complex tetrapeptide that can be divided into four regions, Mep (D-N-methylpipecolinic acid), Ile (isoleucine), Tuv (tubuvaline), and Tup (tubuphenylalanine), as shown in the formula:

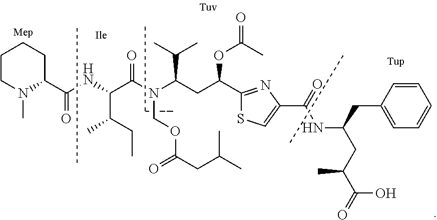

Most of the more potent derivatives of tubulysin, including tubulysin D, also incorporate the interesting O-acyl N,O-acetal functionality, which has rarely been observed in natural products. This reactive functionality is labile in both acidic and basic reaction conditions, and therefore may play a key role in the function of the tubulysins. (Iley et al., Pharm. Res. 1997, 14, 1634-1639). Recently, the total synthesis of tubulysin D was reported, which represents the first synthesis of any member of the tubulysin family that incorporates the O-acyl N,O-acetal functionality. (Peltier et al., J. Am. Chem. Soc. 2006, 128, 16018-16019). Other tubulysins, including tubulysins U and V, have been synthesized by Dömling et al., “Total Synthesis of Tubulysins U and V”, Angew. Chem. Int. Ed. 2006, 45, 7235-7239.

US Patent Application Publication No. US 2011/0021568 A1 (Ellman et al.) discloses the synthesis and activities of a number of tubulysin analogs, including compounds (40) and (10), referred to here as T1 and T2, respectively:

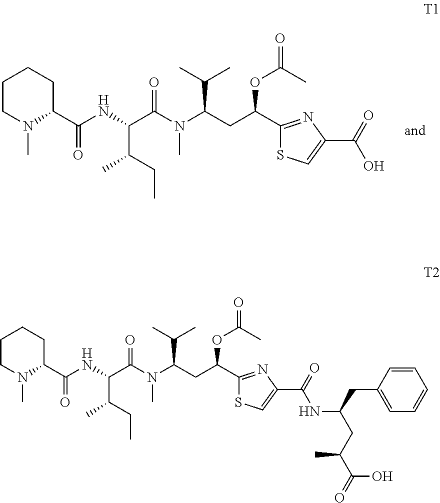 [SMILES: CC[C@H](C)[C@H](NC(=O)[C@H]1CCCCN1C)C(=O)N(C)[C@H](C[C@@H](OC(C)=O)c1nc(C(=O)O)cs1)C(C)C ; CC[C@H](C)[C@H](NC(=O)[C@H]1CCCCN1C)C(=O)N(C)[C@H](C[C@@H](OC(C)=O)c1nc(C(=O)N[C@@H](Cc2ccccc2)C[C@H](C)C(=O)O)cs1)C(C)C]

Schumacher et al., “In Situ Maleimide Bridging of Disulfides and a New Approach to Protein PEGylation”, Bioconjugate Chem. 2011, 22, 132-136, disclose the synthesis of 3,4-disubstituted maleimides such as 3,4-bis(2-hydroxyethylsulfanyl)pyrrole-2,5-dione [referred to by Schumacher et al. as “dimercaptoethanolmaleimide”] and 3,4-bis(phenylsulfanyl)pyrrole-2,5-dione [“dithiophenolmaleimide”], and their N-PEGylated derivatives as PEGylating agents for somatostatin, where the substituted maleimide bonds to the two sulfur atoms of an opened cysteine-cysteine disulfide bond.

It would be desirable to develop potent, homogeneous ADCs, compositions containing them and methods for their use in treating cancers, and methods and intermediates in their preparation.

The disclosures of the documents referred to in this application are incorporated into this application by reference.

### SUMMARY OF THE INVENTION

In a first aspect, this invention is antibody-cytotoxin antibody-drug conjugates (ADCs) of the formula:

A(=PD−L−CTX)ₙ,

where:
A is an antibody,
PD is pyrrole-2,5-dione or pyrrolidine-2,5-dione,
the double bond represents bonds from the 3- and 4-positions of the pyrrole-2,5-dione or pyrrolidine-2,5-dione to the two sulfur atoms of an opened cysteine-cysteine disulfide bond in the antibody,
L is —(CH₂)ₘ— or —(CH₂CH₂O)ₘCH₂CH₂—,
CTX is a cytotoxin bonded to L by an amide bond,
n is an integer of 1 to 4, and
m is an integer of 1 to 12.
Because of the bidentate binding of the PD to the two sulfur atoms of an opened cysteine-cysteine disulfide bond in the antibodies, these ADCs are homogeneous and have enhanced stability over ADCs with monodentate linkers. They will therefore have increased half-lives in vivo, reducing the amount of cytotoxin released systemically, and be safer than ADCs with monodentate linkers.

In a second aspect, this invention is pharmaceutical compositions containing ADCs of the first aspect of this invention; and in a third aspect, this invention is methods of treatment of cancers targeted by the relevant antibodies by administering ADCs of the first aspect of this invention or pharmaceutical compositions of the second aspect of this invention.

In a fourth aspect, this invention is linker-cytotoxin conjugates of formula A, formula B, and formula C:

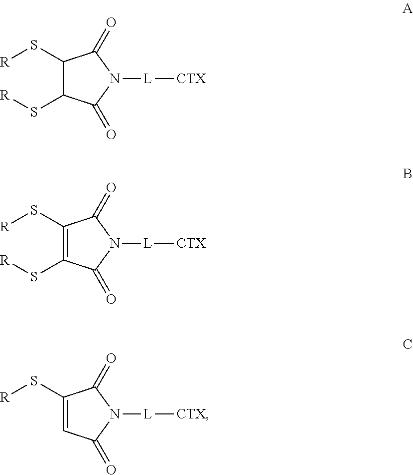

where R is C₁₋₆ alkyl, optionally substituted with halo or hydroxyl; phenyl, optionally substituted with halo, hydroxyl, carboxyl, C₁₋₃ alkoxycarbonyl, or C₁₋₃ alkyl; naphthyl, optionally substituted with halo, hydroxyl, carboxyl, C₁₋₃ alkoxycarbonyl, or C₁₋₃ alkyl; or 2-pyridyl, optionally substituted with halo, hydroxyl, carboxyl, C₁₋₃ alkoxycarbonyl, or C₁₋₃ alkyl,
L is —(CH₂)ₘ— or —(CH₂CH₂O)ₘCH₂CH₂—,
CTX is a cytotoxin bonded to L by an amide bond, and
m is an integer of 1 to 12.
These bidentate linker-cytotoxin conjugates are useful in preparing the antibody-drug conjugates of the first aspect of this invention.

In a fifth aspect, this invention is linkers of formula AA, BB, and CC:

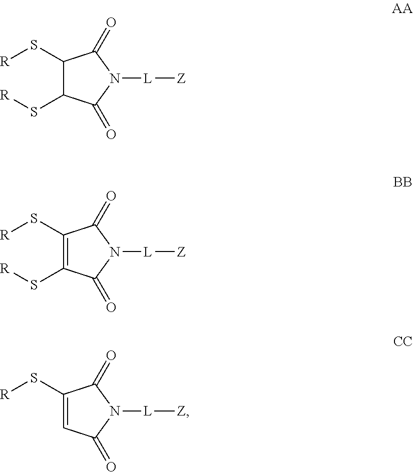

where R is C₁₋₆ alkyl, optionally substituted with halo or hydroxyl; phenyl, optionally substituted with halo, hydroxyl, carboxyl, C₁₋₃ alkoxycarbonyl, or C₁₋₃ alkyl; naphthyl, optionally substituted with halo, hydroxyl, carboxyl, C₁₋₃ alkoxycarbonyl, or C₁₋₃ alkyl; or 2-pyridyl, optionally substituted with halo, hydroxyl, carboxyl, C₁₋₃ alkoxycarbonyl, or C₁₋₃ alkyl,
L is —(CH₂)ₘ— or —(CH₂CH₂O)ₘCH₂CH₂—,
Z is carboxyl, C₁₋₆ alkoxycarbonyl, or amino, and
m is an integer of 1 to 12.
These bidentate linkers are useful in preparing the linker-cytotoxin conjugates of the fourth aspect of this invention.

In a sixth aspect, this invention is linkers of formula AAA, BBB, and CCC:

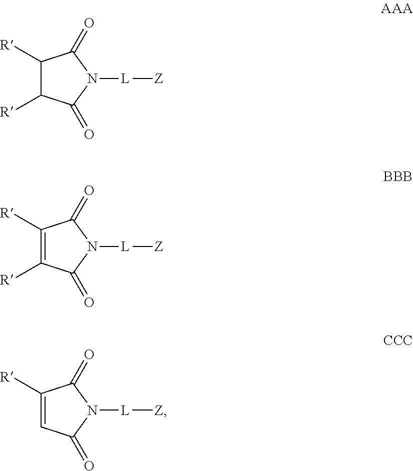

where R′ is chloro, bromo, iodo, C₁₋₆ alkylsulfonyloxy, trifluoromethanesulfonyloxy, benzenesulfonyloxy, or 4-toluenesulfonyloxy,
L is —(CH₂)ₘ— or —(CH₂CH₂O)ₘCH₂CH₂—,
Z is carboxyl, C₁₋₆ alkoxycarbonyl, or amino, and
m is an integer of 1 to 12.
These bidentate linkers are also useful in preparing the linker-cytotoxin conjugates of the fourth aspect of this invention, and are useful in preparing the linkers of the fifth aspect of this invention.

In a seventh aspect, this invention is tubulysins of the formulae of the formulae T3 and T4:

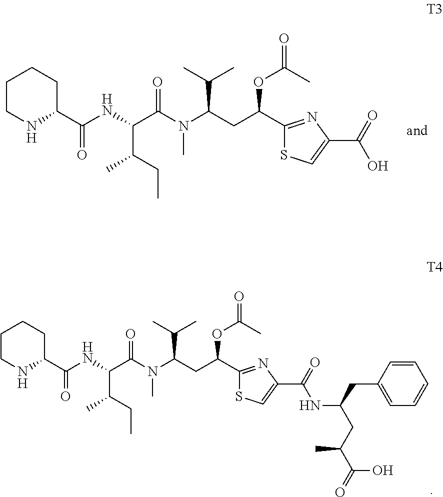 [SMILES: CC[C@H](C)[C@H](NC(=O)[C@H]1CCCCN1)C(=O)N(C)[C@H](C[C@@H](OC(C)=O)c1nc(C(=O)O)cs1)C(C)C ; CC[C@H](C)[C@H](NC(=O)[C@H]1CCCCN1)C(=O)N(C)[C@H](C[C@@H](OC(C)=O)c1nc(C(=O)N[C@@H](Cc2ccccc2)C[C@H](C)C(=O)O)cs1)C(C)C]

These new tubulysins are analogs of the known tubulysins T1 and T2 referred to previously, but because the terminal N-methylpiperidine has been replaced by an unsubstituted piperidine, these new compounds are able to form tubulysin-linker conjugates with linkers containing a carboxyl group by forming an amide bond between the piperidine nitrogen atom and the carbonyl of the linker carboxy group.

Preferred embodiments of this invention are characterized by the specification and by the features of claims 1 to 47 of this application as filed, and of corresponding pharmaceutical compositions, methods, and uses of these compounds.

### DETAILED DESCRIPTION OF THE INVENTION

Definitions

An “antibody”, also known as an immunoglobulin, is a large Y-shaped protein used by the immune system to identify and neutralize foreign objects such as bacteria and viruses. The antibody recognizes a unique part of the foreign target, called an antigen, because each tip of the “Y” of the antibody contains a site that is specific to a site on an antigen, allowing these two structures to bind with precision. An antibody consists of four polypeptide chains, two identical heavy chains and two identical light chains connected by cysteine disulfide bonds. A “monoclonal antibody” is a monospecific antibody where all the antibody molecules are identical because they are made by identical immune cells that are all clones of a unique parent cell. Initially, monoclonal antibodies are typically prepared by fusing myeloma cells with the spleen cells from a mouse (or B-cells from a rabbit) that has been immunized with the desired antigen, then purifying the resulting hybridomas by such techniques as affinity purification. Recombinant monoclonal antibodies are prepared in viruses or yeast cells rather than in mice, through technologies referred to as repertoire cloning or phage display/yeast display, the cloning of immunoglobulin gene segments to create libraries of antibodies with slightly different amino acid sequences from which antibodies with desired specificities may be obtained. The resulting antibodies may be prepared on a large scale by fermentation. “Chimeric” or “humanized” antibodies are antibodies containing a combination of the original (usually mouse) and human DNA sequences used in the recombinant process, such as those in which mouse DNA encoding the binding portion of a monoclonal antibody is merged with human antibody-producing DNA to yield a partially-mouse, partially-human monoclonal antibody. Full-humanized antibodies are produced using transgenic mice (engineered to produce human antibodies) or phage display libraries. Antibodies of particular interest in this invention are those that are specific to cancer antigens, are non-immunogenic, have low toxicity, and are readily internalized by cancer cells; and suitable antibodies include alemtuzumab, bevacizumab, brentuximab, cetuximab, gemtuzumab, ipilimumab, ofatumumab, panitumumab, rituximab, tositumomab, and trastuzumab.

A “cytotoxin” is a molecule that, when released within a cancer cell, is toxic to that cell. Cytotoxins of particular interest in this invention are the tubulysins (such as the tubulysins of the formulae T3 and T4), the auristatins (such as monomethylauristatin E and monomethylauristatin F), the maytansinoids (such as mertansine), the calicheamicins (such as calicheamicin γ); and especially those cytotoxins that, like the tubulysins of the formulae T3 and T4, are capable of coordination through an amide bond to a linker, such as by possessing a basic amine or a carboxyl group.

A “linker” is a molecule with two reactive termini, one for conjugation to an antibody and the other for conjugation to a cytotoxin. The antibody conjugation reactive terminus of the linker is typically a site that is capable of conjugation to the antibody through a cysteine thiol or lysine amine group on the antibody, and so is typically a thiol-reactive group such as a double bond (as in maleimide) or a leaving group such as a chloro, bromo, or iodo, or an R-sulfanyl group, or an amine-reactive group such as a carboxyl group; while the antibody conjugation reactive terminus of the linker is typically a site that is capable of conjugation to the cytotoxin through formation of an amide bond with a basic amine or carboxyl group on the cytotoxin, and so is typically a carboxyl or basic amine group. When the term “linker” is used in describing the linker in conjugated form, one or both of the reactive termini will be absent (such as the leaving group of the thiol-reactive group) or incomplete (such as the being only the carbonyl of the carboxylic acid) because of the formation of the bonds between the linker and/or the cytotoxin.

An “antibody-drug conjugate”, or “ADC” is an antibody that is conjugated to one or more (typically 1 to 4) cytotoxins, each through a linker. The antibody is typically a monoclonal antibody specific to a cancer antigen.

“Tubulysin” includes both the natural products described as tubulysins, such as by Sasse et al. and other authors mentioned in the Description of the related art, and also the tubulysin analogs described in US Patent Application Publication No. US 2011/0021568 A1. Tubulysins of particular interest in this invention are the tubulysins of the formulae T3 and T4, and other tubulysins where the terminal N-methylpiperidine has been replaced by an unsubstituted piperidine, allowing amide bond formation with a linker.

A “basic amine”, such as the amine forming a part of the terminal piperidine group of the tubulysins of the formulae T3 and T4, is a primary or secondary amine that is not part of an amide.

A “therapeutically effective amount” means that amount of an ADC of the first aspect of this invention or composition of the second aspect of this invention which, when administered to a human suffering from a cancer, is sufficient to effect treatment for the cancer. “Treating” or “treatment” of the cancer includes one or more of:
(1) limiting/inhibiting growth of the cancer, i.e. limiting its development;
(2) reducing/preventing spread of the cancer, i.e. reducing/preventing metastases;
(3) relieving the cancer, i.e. causing regression of the cancer,
(4) reducing/preventing recurrence of the cancer; and
(5) palliating symptoms of the cancer.

Cancers of interest for treatment include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, oral cancer, liver cancer, bladder cancer, cancer of the urinary tract, hepatoma, breast cancer including, for example, HER2-positive breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, multiple myeloma and B-cell lymphoma, brain cancer, head and neck cancers, and associated metastases.

Abbreviations/Acronyms

ADC: antibody-drug conjugate; DEA: diethylamine; DCC: 1,3-dicyclohexylcarbodiimide; DIAD: diisopropyl azodicarboxylate; DIPC: 1,3-diisopropylcarbodiimide; DIPEA: diisopropylethylamine; DMF: N,N-dimethylformamide; DPBS: Dulbecco's phosphate-buffered saline; DTPA: diethylenetriaminepentaacetic acid; DTT: dithiothreitol; EDC: ethyl 3-(3-dimethylaminopropyl)carbodiimide; HATU: O-(7-azabenzotriazol-1-yl)-N,N,N′,N′-tetramethyluronium hexafluorophosphate; HOBT: N-hydroxybenzotriazole; NHS: N-hydroxysuccinimide; NMM: N-methylmorpholine; MMAE: monomethylauristatin E; MMAF: monomethylauristatin F, monomethylauristatin phenylalanine; MC: maleimidocaproyl, 6-(2,5-dioxopyrrolyl)hexanoyl; PBS: phosphate-buffered saline; PEG: poly(ethyleneglycol); TBTU: 2-(1H-benzotriazol-1-yl)-1,1,3,3-tetramethyluronium tetrafluoroborate; TCEP: tris(2-carboxyethyl)phosphine; TGI: tumor growth inhibition.

The ADCs of the Invention

As mentioned in the Description of the related art, ADCs of the prior art that coordinate to cysteine thiols of the antibody have employed monofunctional linkers, of which the MC linker is an example. Reduction and opening of the cysteine-cysteine disulfide bonds to give free thiols for conjugation decreases the stability of the antibody, and the formation of the ADC by reaction of the reduced thiols does not re-form a bond, as illustrated in the scheme below:

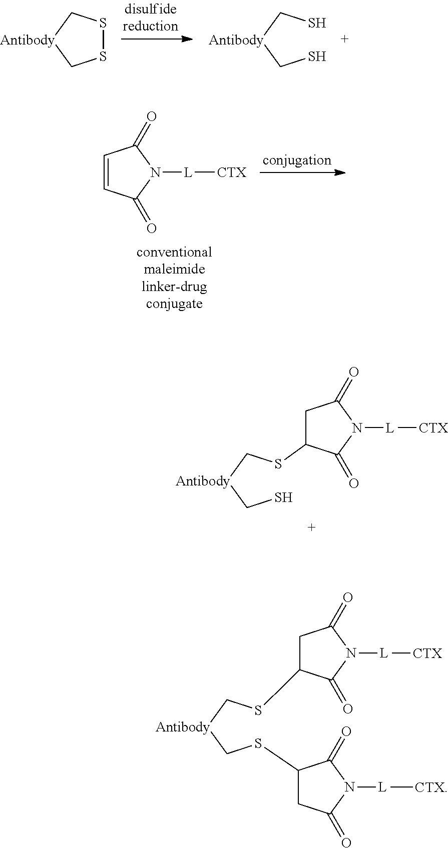

However, the bifunctional pyrrole-2,5-dione- and pyrrolidine-2,5-dione-based linkers of this invention contain two reactive functional groups (X in the scheme below) that react with the two sulfur atoms of an opened cysteine-cysteine disulfide bond. Reaction of the bifunctional linker with the two cysteines gives a “stapled” dithiosuccinimide or dithiomaleimide antibody conjugate with one linker per disulfide connected through two thioether bonds, as shown in the scheme below (double bond absent from the ring: succinimide linkers of formulae AA and AAA; double bond present in the ring: maleimide linkers of formulae BB and BBB):

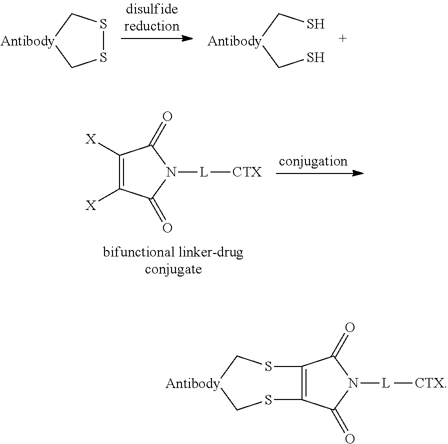

Unlike conventional methods for cysteine conjugation, the reaction re-forms a covalently bonded structure between the 2 cysteine sulfur atoms and therefore does not compromise the overall stability of the antibody. The method also enables conjugation of an optimal 4 drugs per antibody to afford a homogeneous ADC since all of the reactive cysteines are used. The overall result is replacement of a relatively labile disulfide with a stable “staple” between the cysteines. The monosubstituted maleimide linkers (formulae CC and CCC) are also effectively bifunctional in conjugation with the antibody because the double bond of the maleimide is capable of conjugation to one of the cysteine sulfur atoms and the X group with the other.

Preparation of the Compounds of the Invention

The compounds of the invention, such as ADCs, linker-cytotoxin conjugates, linkers, and tubulysins, are prepared by conventional methods of organic and bio-organic chemistry. See, for example, Larock, “Comprehensive Organic Transformations”, Wiley-VCH, New York, N.Y., U.S.A. Suitable protective groups and their methods of addition and removal, where appropriate, are described in Greene et al., “Protective Groups in Organic Synthesis”, 2^{nd} ed., 1991, John Wiley and Sons, New York, N.Y., US. Reference may also be made to the documents referred to elsewhere in the application, such as to the Schumacher et al. article referred to earlier for the synthesis of linkers, US Patent Application Publication No. US 2011/0021568 A1 for the preparation of tubulysins, etc.

Preparation of the Tubulysins

Tubulysins T3 and T4 are prepared by methods analogous to those of Peltier et al. and US Patent Application Publication No. US 2011/0021568 A1, by substituting D-pipecolinic acid for the D-N-methylpipecolinic acid, protecting and deprotecting if appropriate.

Preparation of the Linkers

The comparator MC linker is prepared by methods known to the art for its preparation.

Linkers of this invention are prepared by methods analogous to those of Schumacher et al., as follows (in this reaction scheme, R, L and Z have the meanings given them in the discussion of the fifth and sixth aspects of the invention above):

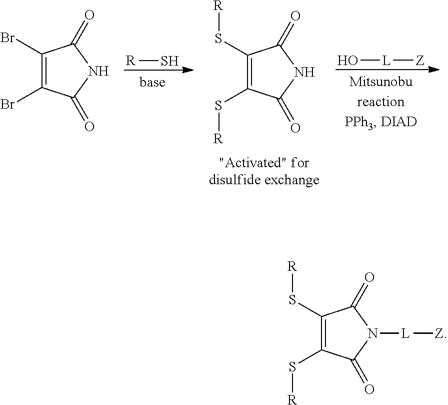

2,3-Dibromomaleimide, 1 equivalent, and a base such as sodium bicarbonate, about 5 equivalents, are dissolved in methanol, and a solution of 2-pyridinethiol, slightly more than 1 equivalent, in methanol, is added. The reaction is stirred for 15 min at ambient temperature. The solvent is removed under vacuum and the residue is purified, such as by flash chromatography on silica gel (petroleum ether:ethyl acetate, gradient elution from 9:1 to 7:3, to give 3,4-bis(2-pyridyl sulfanyl)pyrrole-2,5-dione.

The coupling of the 3,4-bis(2-pyridylsulfanyl)pyrrole-2,5-dione with the sidechain is performed under strictly dry conditions. To the 3,4-bis(2-pyridylsulfanyl)pyrrole-2,5-dione, 1 equivalent, and triphenylphosphine, 1 equivalent, in a mixture of tetrahydrofuran and dichloromethane, is added dropwise DIAD, 1 equivalent, at −78° C. The reaction is stirred for 5 min and the sidechain, 0.5 equivalent, in dichloromethane is added dropwise. After stirring for 5 min, neopentyl alcohol, 1 equivalent, in tetrahydrofuran and dichloromethane is added, and stirred for a further 5 min, then the 3,4-bis(2-pyridylsulfanyl)pyrrole-2,5-dione, 1 equivalent, is added and stirred for another 5 min The reaction is allowed to warm to ambient temperature with stirring for 20 hr, then the solvents are removed under vacuum. The residue is purified, such as by flash chromatography on silica gel (methanol:dichloromethane, gradient elution from 0-10% methanol), to give the linker. The sidechain may be used in protected form, and deprotected following the Mitsunobu reaction, if appropriate.

Alternatively, the sidechain, optionally protected if appropriate, may be coupled to a 3,4-dibromomaleimide by Mitsunobu coupling; and the resulting compound activated for disulfide exchange by reaction with an R-thiol in the presence of base; in the reverse of the synthesis described in the two previous paragraphs.

A similar method may be used for linkers containing the pyrrolidine-2,5-dione moiety rather than the pyrrole-2,5-dione moiety shown above, by starting with 2,3-dibromosuccinimide; but more usually these linkers are prepared by preparing the linker with an unsubstituted maleimide and brominating the linker to give the dibromosuccinimide moiety after coupling with the sidechain, and then “activating” the linker with the R-thiol as a last step.

Mono-substituted maleimide linkers are conveniently prepared by dehydrobromination of the dibromosuccinimide linkers under basic conditions, and related methods.

Preparation of the Linker-Cytotoxin Conjugates

Linker-cytotoxin conjugates may be prepared by methods analogous to those of Doronina et al., Bioconjugate Chem. 2006, 17, 114-124, and similar documents. The linker, 1 equivalent, and HATU, 1 equivalent, are dissolved in anhydrous DMF, followed by the addition of DIPEA, 2 equivalents. The resulting solution is added to the cytotoxin, 0.5 equivalents, dissolved in DMF, and the reaction stirred at ambient temperature for 3 hr. The linker-cytotoxin conjugate is purified by reverse phase HPLC on a C-18 column

Preparation of ADCs

Antibodies, typically monoclonal antibodies are raised against a specific cancer target (antigen), and purified and characterized. Therapeutic ADCs containing that antibody are prepared by standard methods for cysteine conjugation, such as by methods analogous to those of Hamblett et al., “Effects of Drug Loading on the Antitumor Activity of a Monoclonal Antibody Drug Conjugate”, Clin. Cancer Res. 2004, 10, 7063-7070; Doronina et al., “Development of potent and highly efficacious monoclonal antibody auristatin conjugates for cancer therapy”, Nat. Biotechnol., 2003, 21(7), 778-784; and Francisco et al., “cAC10-vcMMAE, an anti-CD30-monomethylauristatin E conjugate with potent and selective antitumor activity”, Blood, 2003, 102, 1458-1465. Antibody-drug conjugates with four drugs per antibody are prepared by partial reduction of the antibody with an excess of a reducing reagent such as DTT or TCEP at 37° C. for 30 min, then the buffer exchanged by elution through SEPHADEX® G-25 resin with 1 mM DTPA in DPBS. The eluent is diluted with further DPBS, and the thiol concentration of the antibody may be measured using 5,5′-dithiobis(2-nitrobenzoic acid) [Ellman's reagent]. An excess, for example 5-fold, of the linker-cytotoxin conjugate is added at 4° C. for 1 hr, and the conjugation reaction may be quenched by addition of a substantial excess, for example 20-fold, of cysteine. The resulting ADC mixture may be purified on SEPHADEX G-25 equilibrated in PBS to remove unreacted linker-cytotoxin conjugate, desalted if desired, and purified by size-exclusion chromatography. The resulting ADC may then be then sterile filtered, for example, through a 0.2 μM filter, and lyophilized if desired for storage.

The formation of an ADC of this invention is illustrated by the reaction scheme below, where the “Y”-shaped structure denotes the antibody, only one disulfide bond is shown, and details of the linker-cytotoxin conjugate are omitted for simplicity in showing the concept of the ADC:

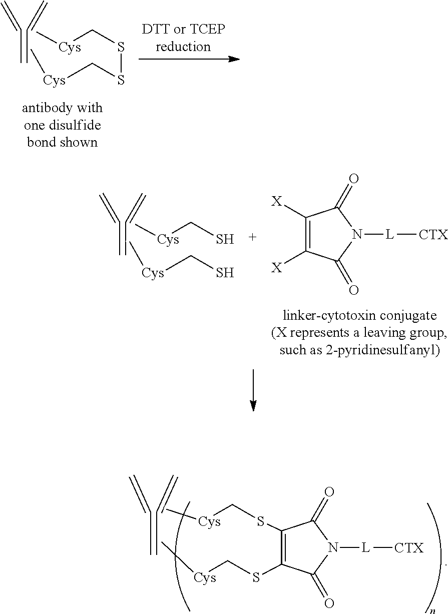

Typically, n will be 4, where all of the interchain cysteine disulfide bonds are replaced by linker-drug conjugates. Schumacher et al. in their conjugation to somatostatin add the reducing agent to a mixture of the somatostatin and the PEGylated linker, so this may be possible with antibodies and linker-cytotoxin conjugates also and is not excluded as a method of synthesis.

Assays

The ADCs of this invention may be assayed for binding affinity to and specificity for the desired antigen by any of the methods conventionally used for the assay of antibodies; and they may be assayed for efficacy as anticancer agents by any of the methods conventionally used for the assay of cytostatic/cytotoxic agents, such as assays for potency against cell cultures, xenograft assays, and the like. A person of ordinary skill in the art will have no difficulty, considering that skill and the literature available, in determining suitable assay techniques; from the results of those assays, in determining suitable doses to test in humans as anticancer agents, and, from the results of those tests, in determining suitable doses to use to treat cancers in humans.

Formulation and Administration

The ADCs of the first aspect of this invention will typically be formulated as solutions for intravenous administration, or as lyophilized concentrates for reconstitution to prepare intravenous solutions (to be reconstituted, e.g., with normal saline, 5% dextrose, or similar isotonic solutions). They will typically be administered by intravenous injection or infusion. A person of ordinary skill in the art of pharmaceutical formulation, especially the formulation of anticancer antibodies, will have no difficulty, considering that skill and the literature available, in developing suitable formulations.

### EXAMPLES

Synthesis of Linkers

### Example 1

### Synthesis of 3,4-bis(2-pyridylsulfanyl)pyrrole-2,5-dione

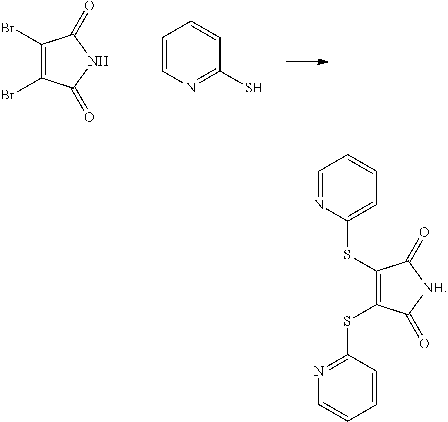 [SMILES: O=C1NC(=O)C(Br)=C1Br ; Sc1ccccn1 ; O=C1NC(=O)C(Sc2ccccn2)=C1Sc1ccccn1]

3,4-Dibromopyrrole-2,5-dione [2,3-dibromomaleimide], 1 g, was added to a clean 100 mL round bottom flask with a rubber stopper and bubbler, and dissolved in 50 mL HPLC grade methanol. 2-Pyridinethiol, 2 equivalents, was added to a 20 mL scintillation vial, and dissolved in 10 mL methanol. Under nitrogen and with stirring, the 2-pyridinethiol/methanol solution was added dropwise to the 3,4-dibromopyrrole-2,5-dione via a 20 mL syringe with a 16 gauge needle, and the reaction mixture was stirred for an additional 3-4 hours. The methanol was evaporated and the crude product was dissolved in ethyl acetate and loaded onto about 2 g silica gel. The silica gel-loaded crude product was eluted through a 12 g silica gel cartridge with a hexane:ethyl acetate gradient from 9:1 to 0:1 over 25 column volumes. The enriched fractions were identified, pooled and lyophilized to dryness. The final product was recrystallized from ethyl acetate and diethyl ether to provide yellow needle crystals which were collected by filtration.

Similar syntheses may be performed using the methods of Schumacher et al. for other 3,4-di(R-sulfanyl)pyrrole-2,5-diones (see the Supplementary Materials at pages S17-S18). Similar syntheses may also be performed starting with (3,4-dibromo-2,5-dioxopyrrolyl)-terminated linkers [i.e. compounds where a sidechain has already been added to the pyrrole nitrogen] to give the corresponding (2,5-dioxo-3,4-di(R-sulfanyl)pyrrolyl)-terminated linkers; and/or with other thiols (such as the benzenethiol and 2-hydroxyethanethiol of Schumacher et al.) to give the corresponding linkers; and/or with other pyrrolediones or pyrrolidinediones, such as 3,4-dichloropyrrole-2,5-dione or 3,4-dibromopyrrolidine-2,5-dione, or based on them, to give the corresponding 3,4-di(R-sulfanyl)pyrrole-2,5-diones or 3,4-di(R-sulfanyl)pyrrolidine-2,5-diones or linkers based on them.

### Example 2

### Synthesis of 39-(3,4-dibromo-2,5-dioxopyrrolyl)-3,6,9,12,15,18,21,24,27,30,33,36-dodecaoxanonatriacontanoic Acid:

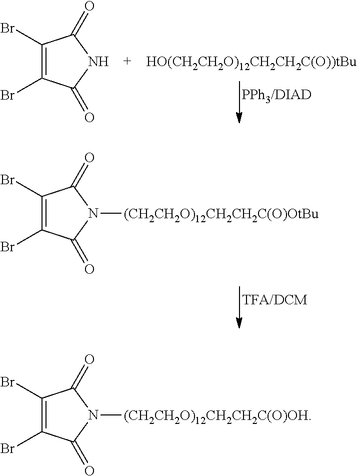

A 100 mL two-necked round bottom flask was flame dried and cooled under nitrogen. The cooled flask was charged with 200 mg (0.296 mmol) of tert-butyl 39-hydroxy-3,6,9,12,15,18,21,24,27,30,33,36-dodecaoxanonatriacontanoate. Triphenylphosphine, 106 mg, was dissolved in about 5 mL anhydrous tetrahydrofuran in a vial, and the solution was added to the 100 mL flask via cannula under nitrogen. The 100 mL flask was cooled in an ice-water bath for 15 minutes. To the cooled solution was added 55 mg (0.217 mmol) 3,4-dibromopyrrole-2,5-dione with stirring until a clear solution was observed. DIAD, 58.3 μL, was added to the cooled reaction mixture, which was stirred in the ice bath for an additional 10 minutes. The reaction mixture was stirred and allowed to reach room temperature over about 20 hours, then concentrated on a rotary evaporator until dry, giving a yellow viscous oil, which was absorbed onto about 1 g silica gel and dry-loaded onto a Reveleris normal phase chromatography unit. The oil was eluted over a 12 g silica gel cartridge with a methanol:dichloromethane gradient from 1:0 to 9:1 over 28 column volumes. The fractions containing the desired product were pooled and concentrated to dryness. The purified product was suspended in 50:50 acetonitrile:water and lyophilized overnight to provide a clear light yellow viscous oil. By LC-MS analysis, the of tert-butyl-protected carboxylic acid product had been partially deprotected during the work-up. To fully deprotect the material to the free acid, the lyophilized material was treated with 5% trifluoroacetic acid in dichloromethane, concentrated to dryness and lyophilized in acetonitrile:water (50:50) overnight.

Similar syntheses may be performed starting with 3,4-bis(2-pyridylsulfanyl)pyrrole-2,5-dione to give 39-(2,5-dioxo-3,4-bis(2-pyridylsulfanyl)pyrrolyl)-3,6,9,12,15,18,21,24,27,30,33,36-dodecaoxanonatriacontanoic acid, or starting with other 3,4-di(R-sulfanyl)pyrrole-2,5-diones to give the corresponding linkers; and/or starting with other hydroxyl-terminated sidechains, e.g. using tert-butyl 6-hydroxyhexanoate to give 6-(3,4-dibromo-2,5-dioxopyrrolyl)hexanoic acid, etc. Similar syntheses starting with maleimide rather than 2,3-dibromomaleimide give comparator linkers of the prior art, such as 6-(2,5-dioxopyrrolyl)hexanoic acid, the MC linker.

### Example 3

### Synthesis of 39-(3,4-dibromo-2,5-dioxopyrrolidinyl)-3,6,9,12,15,18,21,24,27,30,33,36-dodecaoxanonatriacontanoic Acid [the dBrPEG Linker]:

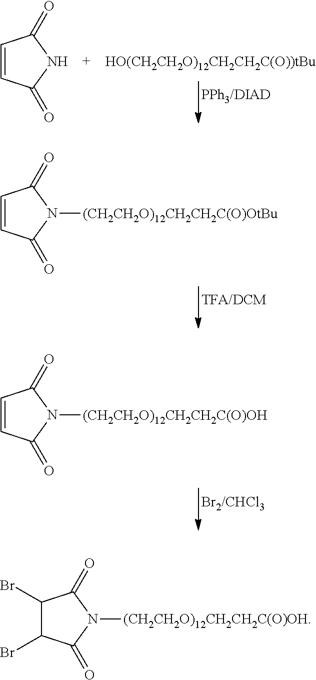

39-(2,5-dioxopyrrolyl)-3,6,9,12,15,18,21,24,27,30,33,36-dodecaoxanonatriacontanoic acid was prepared in the same manner as the 39-(3,4-dibromo-2,5-dioxopyrrolyl)-3,6,9,12,15,18,21,24,27,30,33,36-dodecaoxanonatriacontanoic acid of Example 2, but starting with maleimide rather than 2,3-dibromomaleimide. The acid was treated with 0.5 equivalents of bromine in chloroform followed by refluxing overnight to give 39-(3,4-dibromo-2,5-dioxopyrrolidinyl)-3,6,9,12,15,18,21,24,27,30,33,36-dodecaoxanonatriacontanoic acid after flash purification on silica gel.

Similar syntheses may be performed using other hydroxyl-terminated sidechains, e.g. using tert-butyl 6-hydroxyhexanoate to give 6-(3,4-dibromo-2,5-dioxopyrrolidinyl)hexanoic acid, etc. The dibrominated linkers that are products of this synthesis may be dehydrobrominated with base in an additional step to give (3-bromo-2,5-dioxopyrrolyl)-terminated linkers, such as 6-(3-bromo-2,5-dioxopyrrolyl)hexanoic acid.

Synthesis of Linker-Cytotoxin Conjugates

### Example 4

### Synthesis of T4

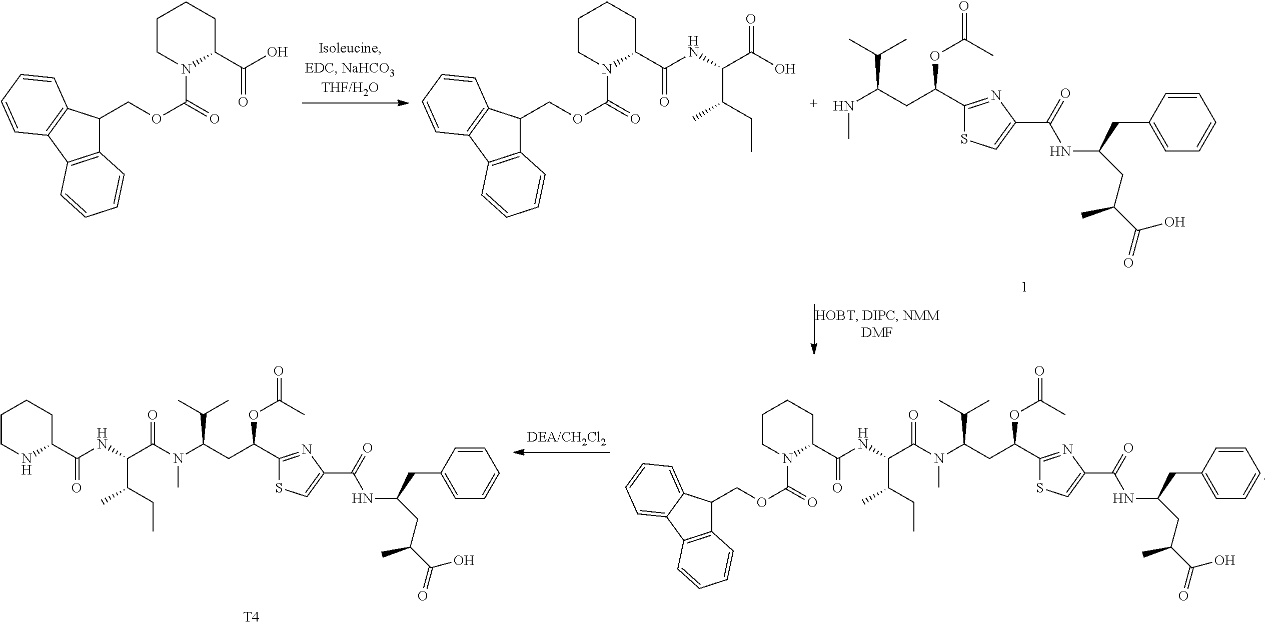 [SMILES: O=C(O)[C@H]1CCCCN1C(=O)OCC1c2ccccc2-c2ccccc21 ; CC[C@H](C)[C@H](NC(=O)[C@H]1CCCCN1C(=O)OCC1c2ccccc2-c2ccccc21)C(=O)O ; CN[C@H](C[C@@H](OC(C)=O)c1nc(C(=O)N[C@@H](Cc2ccccc2)C[C@H](C)C(=O)O)cs1)C(C)C ; CC[C@H](C)[C@H](NC(=O)[C@H]1CCCCN1)C(=O)N(C)[C@H](C[C@@H](OC(C)=O)c1nc(C(=O)N[C@@H](Cc2ccccc2)C[C@H](C)C(=O)O)cs1)C(C)C ; CC[C@H](C)[C@H](NC(=O)[C@H]1CCCCN1C(=O)OCC1c2ccccc2-c2ccccc21)C(=O)N(C)[C@H](C[C@@H](OC(C)=O)c1nc(C(=O)N[C@@H](Cc2ccccc2)C[C@H](C)C(=O)O)cs1)C(C)C ; "T4"]

Fmoc-T4 was prepared by coupling Fmoc-D-2-piperidinecarboxylic acid to isoleucine in the presence of EDC and sodium bicarbonate, then coupling the resulting Fmoc-D-Pip-Ile-OH to the N-methylvaline intermediate 1 (purchased from Concortis) by mixing with 1 equivalent of HOBT and DIPC in DMF followed by addition of 2.5 equivalents of NMM. The reaction mixture was stirred overnight and purified by flash chromatography on silica gel using a gradient of hexane and ethyl acetate. Evaporation of solvent gave Fmoc-T4 as a yellow oil. The Fmoc-T4 was then deprotected by treatment with 20% DEA in methylene chloride for 30 minutes to give T4, which was purified by preparative HPLC on a C18 reverse phase column eluted with acetonitrile/water.

### Example 5

### Synthesis of 6-(2,5-dioxopyrrolyl)hexanoyl-T4 [MC-T4] and 39-(3,4-dibromo-2,5-dioxopyrrolidinyl)-3,6,9,12,15,18,21,24,27,30,33,36-dodecaoxanonatriacontanoyl-T4 [dBrPEG-T4]

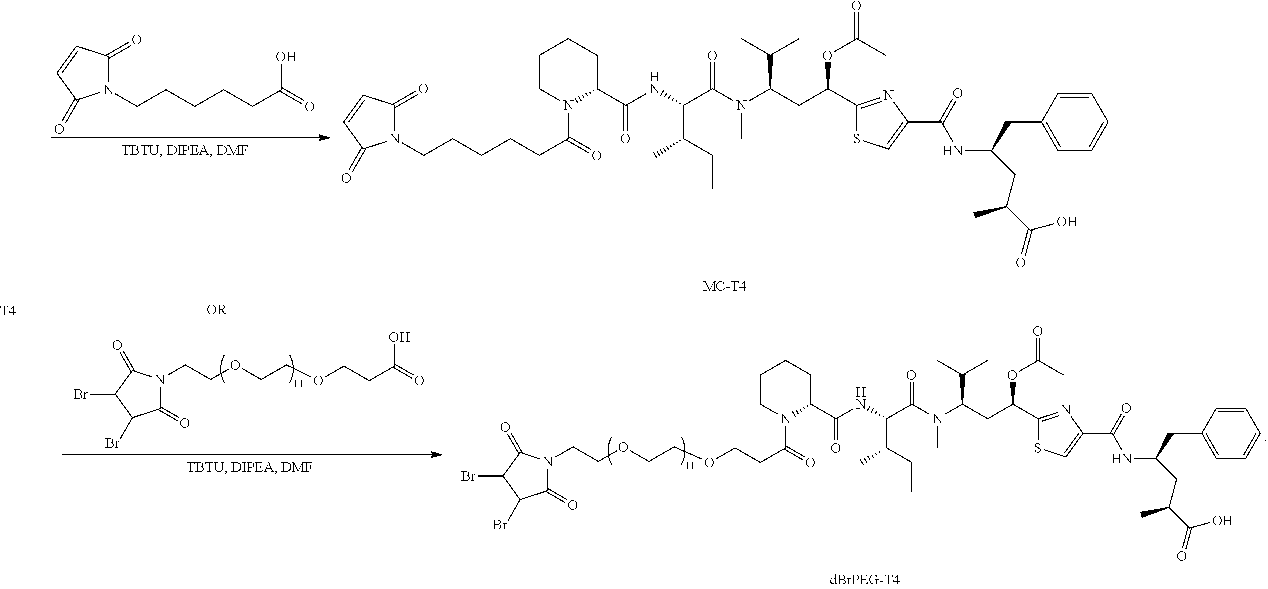

Coupling of T4 to the MC or dBrPEG linkers described in Example 2 and 3 respectively was performed by activating the linkers with 1 equivalent of TBTU in the presence of 2 equivalents of DIPEA in DMF, then coupling with the T4 for 72 hours at room temperature. Purification by preparative C18 HPLC (acetonitrile-water gradient) gave MC-T4 or dBrPEG-T4 suitable for conjugation to antibodies.

Similar syntheses using other linkers give the corresponding linker-T4 conjugates. Similar syntheses using T3, MMAF, or other cytotoxins with a basic amine give the corresponding linker-cytotoxin conjugates. Similar syntheses using amine-terminated linkers and cytotoxins with a carboxyl group, activating the cytotoxin in the same manner as the linker was activated in the above Example, give other linker-cytotoxin conjugates.

### Example 6

### Synthesis of 39-(2,5-dioxo-3,4-bis(2-pyridylsulfanyl)pyrrolyl)-3,6,9,12,15,18,21,24,27,30,33,36-dodecaoxanonatriacontanoyl-MMAF [dPSPEG-MMAF]

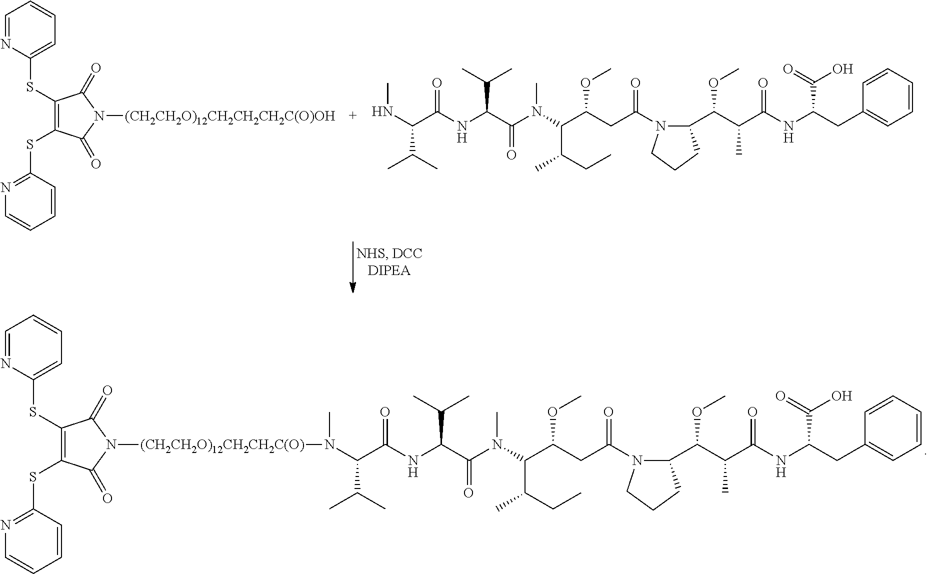

39-(2,5-Dioxo-3,4-bis(pyridin-2-ylthio)-2,5-dihydro-1H-pyrrol-1-yl)-3,6,9,12,15,18,21,24,27,30,33,36-dodecaoxanonatriacontanoic acid was added to a clean, flame-dried 50 mL round bottom flask, and the carboxylic acid was activated with NHS in 3 mL of DMF in the presence of DCC. MMAF was predissolved in about 1 mL DMF and transferred to the NHS-activated acid via 22 gauge needle. DIPEA was added to the reaction mixture and stirred overnight. The crude reaction mixture was purified by reverse-phase HPLC on a 21.2 mm×50 mm Agilent PREP-C18 column at a flow rate of 35 mL/min over 20 column volumes (about 30 minutes of gradient time). Enriched fractions were identified, pooled and lyophilized to give the dPSPEG-MMAF conjugate as a white semi-solid.

Similar syntheses using other linkers give the corresponding linker-MMAF conjugates. Similar syntheses using T3, T4, or other cytotoxins with a basic amine give the corresponding linker-cytotoxin conjugates, such as dPSPEG-T4. Similar syntheses using amine-terminated linkers and cytotoxins with a carboxyl group, activating the cytotoxin in the same manner as the linker was activated in the above Example, give other linker-cytotoxin conjugates.

Synthesis of Antibody-Drug Conjugates

### Example 7

### Synthesis of trastuzumab-dTSPEG-MMAF ADC

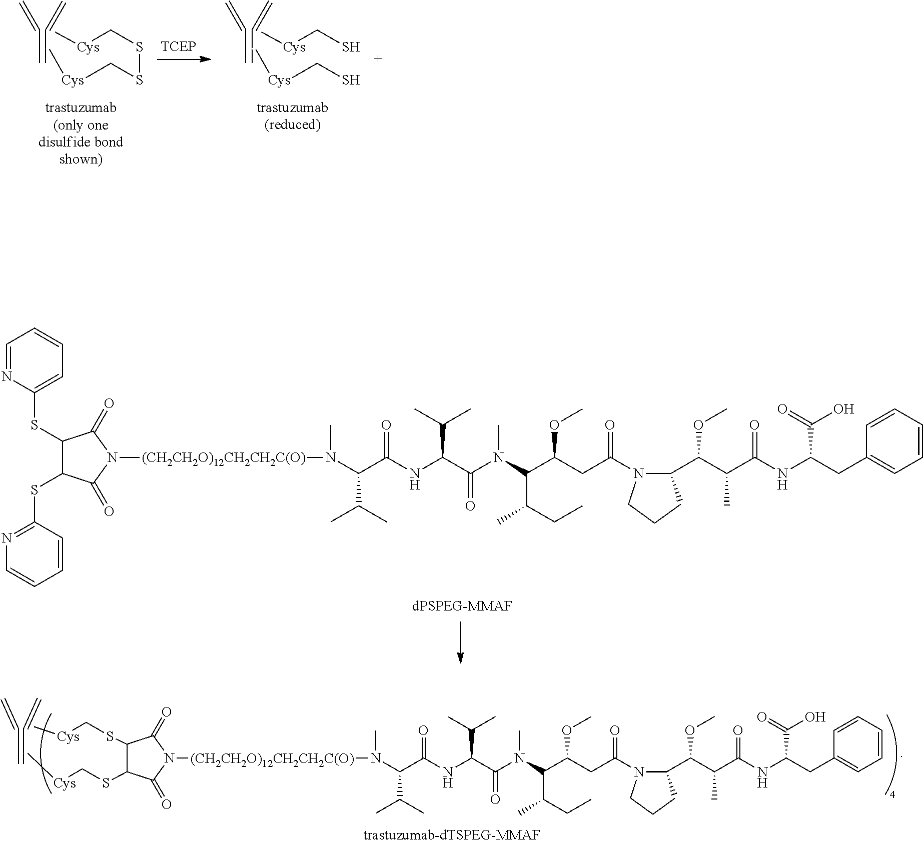

Trastuzumab, 1 mL of a 20 mg/mL solution in pH 7.4 PBS (Gibco Mg and Ca free) with 1 mM DTPA, is loaded into a sterile 1.7 mL Eppendorf tube, then 2.75 equivalents of TCEP hydrochloride (Sigma ampule 0.5M concentration), is added and the mixture incubated at 37° C. for 1 hour to give an average of 4 free thiol pairs per trastuzumab (this can be verified by Ellman's colorimetric assay—see Ellman, “Tissue sulfhydryl groups”, Arch. Biochem. Biophys, 1959, 82, 70-77 or later papers referring to this assay). The reduced antibody solution is cooled in an ice-bath at about 0° C. for 15 minutes; then a solution of about 4 equivalents of dPSPEG-MMAF in dimethylsulfoxide is added and the mixture incubated at 37° C. for 2 hours (or at 4° C. for 20 hours). The resulting trastuzumab-dTSPEG-MMAF ADC is purified by size-exclusion chromatography (GE ÄKTA pure chromatographic system) or PD10 desalting column.

Similar syntheses using other linker-cytotoxin conjugates, such as dPSPEG-T4, and/or other antibodies, such as 18-2A (a murine IgG2a antibody), give the corresponding ADCs.

Assays

ADCs of this invention are tested for potency and selectivity in vitro by determining their cytotoxicity in cancer cell lines of interest, such as those cancer cell lines expressing the antigen corresponding to the antibody portion of the ADC and similar cancer cell lines lacking the antigen. They are tested for potency and safety in vivo in such animal models as the mouse subcutaneous cancer xenograft and mouse orthotopic cancer xenograft models well known to those of skill in the art of cancer research.

### Example 8

### Cytotoxicity of Trastuzumab ADCs Compared to Trastuzumab

The cytotoxicity of two ADCs where trastuzumab was conjugated to the currently used cytotoxin MMAF through an MC linker [trastuzumab-MC-MMAF] was compared to the cytotoxicity of trastuzumab alone in HER2-positive and HER2-negative tumor cells. In the HER2-negative tumor cells, the IC₅₀ for both ADCs and for trastuzumab itself was >500 nM; however, in the HER2-positive tumor cells, while the IC₅₀ for trastuzumab itself was still >500 nM, the two trastuzumab-MC-MMAF ADCs had IC₅₀s of 0.009 nM and 0.018 nM. These results suggest that ADCs are considerably more potent than their parental antibodies.

### Example 9

### Cytotoxicity of T1 and T2 Compared to MMAF

The cytotoxicity of tubulysins T1 and T2 was compared to the cytotoxicity of MMAF using the BT474 (HER2+) cell line in a standard cellular cytotoxicity assay. In these cells, MMAF had an IC₅₀ of 93 nM, T1 had an IC₅₀ of 11 nM, and T2 had an IC₅₀ of <0.1 nM, showing that these tubulysins are considerably more potent than MMAF. These results suggest that that the N-conjugable tubulysins T3 and T4 are of similar potency to non-N-conjugable tubulysins T1 and T2, and considerably more potent than MMAF. These results and the results of Example 8 suggest that tubulysin ADCs are considerably more potent than MMAF ADCs, and will be effective anticancer agents.

### Example 10

### Binding Affinity of ADCs for Antigen-Expressing Cells

Binding of the antibodies and ADCs to antigen-expressing cells are measured using a cell ELISA. Sarcoma cells transduced to express the target (F279 cells for HER2, F244 cells for CD98) are plated the day at 5000 cells per well in a 384-well plate. The following day, antibodies are serially diluted in a separate plate, and then transferred to the cell plate, which has previously had media removed by aspiration. After a 2 hour incubation at room temperature, the plate is washed with wash buffer (DPBS at pH7.4 with 0.1% bovine serum albumin) and then 25 μL horseradish peroxidase-labeled secondary antibody diluted in media is added and incubated for 30 minutes at room temperature. The plate is then washed and 15 μL of a chemiluminescent substrate (Pierce catalog #37069) is added; and the plate is read in a plate-based luminescence reader. Trastuzumab and trastuzumab ADCs (trastuzumab-MC-MMAF, trastuzumab-MC-T4, trastuzumab-dTSPEG-MMAF, and trastuzumab-dTSPEG-T4) demonstrated comparable affinity for F277 cells; and 18-2A and 18-2A ADCs (18-2A-MC-MMAF, 18-2A-MC-T4, 18-2A-dTSPEG-MMAF, and 18-2A-dTSPEG-T4) demonstrated comparable affinity for F244 cells, indicating that conjugation of the drug payloads do not effect antigen binding.

### Example 11

### Potency of ADCs Against Antigen-Expressing Cells

The potency of ADCs for inhibition of tumor cell growth was tested in cell proliferation assays. The Ramos (B-cell lymphoma) and BT474 (HER2+human breast carcinoma) cell lines were seeded into 96 well half-area plates the day before drug treatment at 3000 and 5000 cells per well respectively. ADCs and controls were serially diluted in a master plate, and then transferred to the cell plates, which were incubated at 37 degrees Celsius and 5% CO₂ for 3 days. The cells were quantitated by measuring the level of ATP in the wells using the ATPLite 1Step kit (Perkin Elmer catalog #50-904-9883) as described by the manufacturer. The 18-2A ADCs (18-2A-MC-MMAF, 18-2A-MC-T4, 18-2A-dTSPEG-MMAF, and 18-2A-dTSPEG-T4) were approximately equipotent and considerably more potent than the parent 18-2A antibody in Ramos cells, while the trastuzumab ADCs (trastuzumab-MC-MMAF, trastuzumab-MC-T4, trastuzumab-dTSPEG-MMAF, and trastuzumab-dTSPEG-T4) were approximately equipotent and considerably more potent than the parent trastuzumab antibody in BT474 cells.

### Example 12

### Efficacy of ADCs in Murine Xenograft Models

The Ramos Cell Xenograft Model.

The Ramos cell line was obtained from ATCC and cultured according to the supplier's protocols. 4-6 Week-old immunodeficient female mice (Taconic C.B-17 scid) were subcutaneously injected on the right flank with 1×10⁷ viable cells in a mixture of PBS (without magnesium or calcium) and BD Matrigel (BD Biosciences) at a 1:1 ratio. The injected total volume per mouse was 200 μL with 50% being Matrigel. Once the tumor reached a size of 65-200 mm³, mice were randomized. ADCs were formulated in PBS and administered once intravenously at a dose of 1 mg/Kg into the lateral tail vein, and body weights and tumors were measured twice weekly. Tumor volume was calculated as described in van der Horst et al., “Discovery of Fully Human Anti-MET Monoclonal Antibodies with Antitumor Activity against Colon Cancer Tumor Models In Vivo”, Neoplasia, 2009, 11, 355-364. The experiments were performed on groups of 8 animals per experimental point. The negative control group received HB121 (an IgG2a-negative antibody) and free MMAF or T4, as appropriate, at a concentration equimolar to the concentration that would be released by the ADCs, while the positive control group received 18-2A. The 18-2A ADCs with the linkers of this invention (18-2A-dTSPEG-MMAF and 18-2A-dTSPEG-T4) demonstrated slightly more but comparable TGI than the comparator ADCs (18-2A-MC-MMAF and 18-2A-MC-T4, respectively), and more TGI than the parent 18-2A antibody, while all demonstrated significant TGI compared to the control. No toxicity was observed based on animal weight measurements.

The BT474 Cell Xenograft Model.

The BT474 cell line was obtained from ATCC and cultured according to the supplier's protocols. 4-6 Week-old immunodeficient female mice (Taconic C.B-17 scid) were implanted with a β-estradiol pellet 3 days before being subcutaneously injected on the right flank with 1×10⁷ viable cells in a mixture of PBS (without magnesium or calcium) and BD Matrigel (BD Biosciences) at a 1:1 ratio. The injected total volume per mouse was 200 μL with 50% being Matrigel. Once the tumor reached a size of 100-150 mm³, mice were randomized ADCs were formulated in PBS and administered once intravenously at a dose of 1 mg/Kg into the lateral tail vein, and body weights and tumors were measured twice weekly. Tumor volume was calculated as described in van der Horst et al., cited above. The experiments were performed on groups of 8 animals per experimental point. The negative control group received HB121 and free MMAF or T4, as appropriate, at a concentration equimolar to the concentration that would be released by the ADCs, while the positive control group received trastuzumab at 1 mg/Kg. The trastuzumab ADCs with the linkers of this invention (trastuzumab-dTSPEG-MMAF and trastuzumab-dTSPEG-T4) demonstrated comparable TGI to than the comparator ADCs (trastuzumab-MC-MMAF and trastuzumab-MC-T4, respectively), and slightly more TGI than the parent trastuzumab, while all demonstrated significant TGI compared to the control. No toxicity was observed based on animal weight measurements.

Similar tests are conducted with other cancers (those expressing different antigens) and ADCs where the antibody corresponds to the antigen expressed by the cancer.

While this invention has been described in conjunction with specific embodiments and examples, it will be apparent to a person of ordinary skill in the art, having regard to that skill and this disclosure, that equivalents of the specifically disclosed materials and methods will also be applicable to this invention; and such equivalents are intended to be included within the following claims.

What is claimed is:

1. An antibody-drug conjugate of the formula:
A(=PD−L−CTX)ₙ,
where:
A is an antibody,
PD is pyrrole-2,5-dione or pyrrolidine-2,5-dione,
the double bond represents bonds from the 3- and 4-positions of the pyrrole-2,5-dione or pyrrolidine-2,5-dione to the two sulfur atoms of an opened cysteine-cysteine disulfide bond in the antibody,
L is —(CH₂)ₘ— or —(CH₂CH₂O)ₘCH₂CH₂—,
CTX is a cytotoxin bonded to L by an amide bond,
n is an integer of 1 to 4, and
m is an integer of 1 to 12.

2. The antibody-drug conjugate of claim 1 where A is a monoclonal antibody and CTX is a cytotoxin.

3. The antibody-drug conjugate of claim 1 where A is a human or humanized antibody.

4. The antibody-drug conjugate of claim 1 where A is an antibody that is specific to a cancer antigen.

5. The antibody-drug conjugate of claim 1 where A is alemtuzumab, bevacizumab, brentuximab, cetuximab, gemtuzumab, ipilimumab, ofatumumab, panitumumab, rituximab, tositumomab, or trastuzumab.

6. The antibody-drug conjugate of claim 1 where A is trastuzumab.

7. The antibody-drug conjugate of claim 1 where CTX is an auristatin, a calicheamicin, a maytansinoid, or a tubulysin.

8. The antibody-drug conjugate of claim 7 where CTX is monomethylauristatin E, monomethylauristatin F, calicheamicin γ, mertansine, tubulysin T3, or tubulysin T4.

9. The antibody-drug conjugate of claim 1 where PD is pyrrolidine-2,5-dione.

10. The antibody-drug conjugate of claim 1 where PD is pyrrole-2,5-dione.

11-12. (canceled)

13. A pharmaceutical composition containing an antibody-drug conjugate of claim 1.

14. A method of treating a cancer by administering to a human suffering therefrom an effective amount of an antibody-drug conjugate of claim 1 or a pharmaceutical composition of claim 13.

15. A linker-cytotoxin conjugate of formula A, B, or C: 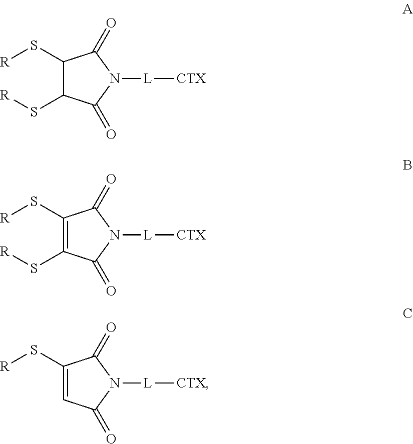
where R is C₁₋₆ alkyl, optionally substituted with halo or hydroxyl; phenyl, optionally substituted with halo, hydroxyl, carboxyl, C₁₋₃ alkoxycarbonyl, or C₁₋₃ alkyl; naphthyl, optionally substituted with halo, hydroxyl, carboxyl, C₁₋₃ alkoxycarbonyl, or C₁₋₃ alkyl; or 2-pyridyl, optionally substituted with halo, hydroxyl, carboxyl, C₁₋₃ alkoxycarbonyl, or C₁₋₃ alkyl,
L is —(CH₂)ₘ— or —(CH₂CH₂O)ₘCH₂CH₂—,
CTX is a cytotoxin bonded to L by an amide bond, and
m is an integer of 1 to 12.

16.-18. (canceled)

19. The linker-cytotoxin conjugate of claim 15 where R is 2-pyridyl, optionally substituted with halo, hydroxyl, carboxyl, C₁₋₃ alkoxycarbonyl, or C₁₋₃ alkyl.

20. (canceled)

21. The linker-cytotoxin conjugate of claim 15 where CTX is an auristatin, a calicheamicin, a maytansinoid, or a tubulysin.

22. The linker-cytotoxin conjugate of claim 21 where CTX is monomethylauristatin E, monomethylauristatin F, calicheamicin γ, mertansine, tubulysin T3, or tubulysin T4.

23. The linker-cytotoxin conjugate of claim 21 where L is —(CH₂)ₘ— or —(CH₂CH₂O)ₘCH₂CH₂—.

24. (canceled)

25. A linker of formula AA, BB, or CC: 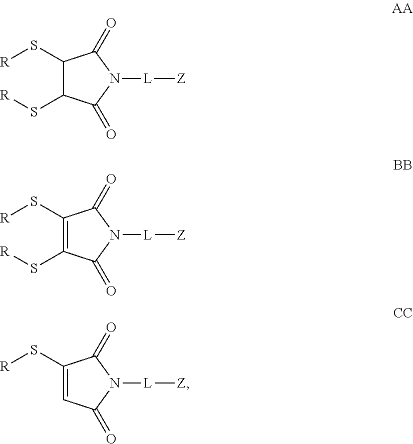
where R is C₁₋₆ alkyl, optionally substituted with halo or hydroxyl; phenyl, optionally substituted with halo, hydroxyl, carboxyl, C₁₋₃ alkoxycarbonyl, or C₁₋₃ alkyl; naphthyl, optionally substituted with halo, hydroxyl, carboxyl, C₁₋₃ alkoxycarbonyl, or C₁₋₃ alkyl; or 2-pyridyl, optionally substituted with halo, hydroxyl, carboxyl, C₁₋₃ alkoxycarbonyl, or C₁₋₃ alkyl,
L is —(CH₂)ₘ— or —(CH₂CH₂O)ₘCH₂CH₂—,
Z is carboxyl, C₁₋₆ alkoxycarbonyl, or amino, and
m is an integer of 1 to 12.

26-34. (canceled)

35. A linker of formula AAA, BBB, or CCC: 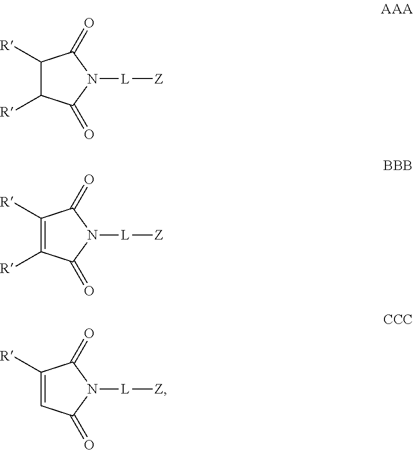
where R′ is chloro, bromo, iodo, C₁₋₆ alkylsulfonyloxy, trifluoromethanesulfonyloxy, benzenesulfonyloxy, or 4-toluenesulfonyloxy,
L is —(CH₂)ₘ— or —(CH₂CH₂O)ₘCH₂CH₂—,
Z is carboxyl, C₁₋₆ alkoxycarbonyl, or amino, and
m is an integer of 1 to 12.

36-45. (canceled)

46. A tubulysin compound of the formula T3:

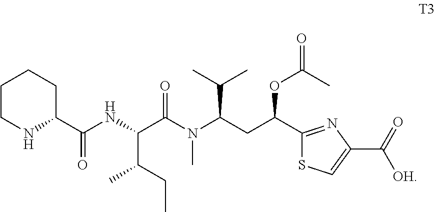 [SMILES: CC[C@H](C)[C@H](NC(=O)[C@H]1CCCCN1)C(=O)N(C)[C@H](C[C@@H](OC(C)=O)c1nc(C(=O)O)cs1)C(C)C]

47. A tubulysin compound of the formula T4: 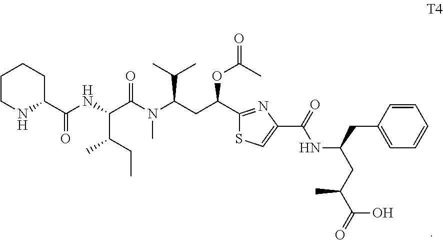 [SMILES: CC[C@H](C)[C@H](NC(=O)[C@H]1CCCCN1)C(=O)N(C)[C@H](C[C@@H](OC(C)=O)c1nc(C(=O)N[C@@H](Cc2ccccc2)C[C@H](C)C(=O)O)cs1)C(C)C]
